(19) United States
(12) Patent Application Publication
(10) Pub. No.: US 2004/0141875 A1
(43) Pub. Date: Jul. 22, 2004

(54) System and method for treating microorganisms within motor vehicle heating, ventilation, and air conditioning units

(76) Inventor: Rajiv Doshi, San Francisco, CA (US)

Correspondence Address: GRAY CARY WARE & FREIDENRICH LLP, 2000 UNIVERSITY AVENUE, E. PALO ALTO, CA, 94303-2248, US

(21) Appl. No.: 10/756,214

(22) Filed: Jan. 12, 2004

Related U.S. Application Data
(60) Provisional application No. 60/440,756, filed on Jan. 15, 2003.

(51) Int. Cl. A61L 9/20

(52) U.S. Cl. 422/4; 422/121; 422/24

(57) ABSTRACT

An HVAC system for a motor vehicle including one or more ultraviolet light sources that are operatively disposed within one or more portions of the HVAC system and that are effective in treating microorganisms within the HVAC system. The HVAC system may also include one or more sensors and a controller for selectively activating and deactivating the one or more ultraviolet light sources.

### CROSS-REFERENCE TO RELATED APPLICATIONS

This application claims priority of U.S. provisional application serial No. 60/440,756 filed on Jan. 15, 2003, which is fully and completely incorporated herein by reference.

### FIELD OF THE INVENTION

This invention generally relates to heating, ventilation, and air conditioning (HVAC) systems within motor vehicles, and more specifically to a system and method for reducing the number of microorganisms in HVAC systems.

### BACKGROUND OF THE INVENTION

Heating, ventilation, and air conditioning (HVAC) systems are a ubiquitous component of motor vehicles. Typically, HVAC systems comprise heating, refrigeration (or air conditioning), and air distribution components. Despite many technologic advances in the field, current HVAC systems still have considerable deficiencies. These systems are not well equipped to prevent the growth of microorganisms (including bacteria, fungi, molds, spores, viruses, and protozoa) within these systems. HVAC systems are also ill suited to prevent transmission of microorganisms from outside the vehicle into the passenger compartment. Finally, current HVAC systems are incapable of cleansing air from the passenger compartment of microorganisms that may be infectious.

The growth of fungus, bacteria, spores, molds, algae, protozoa and other microorganisms on the evaporator and other parts of an HVAC system in a motor vehicle remains a significant problem. As air passes over the fins or coils of the evaporator, the air is both cooled and dehumidified. During the process, moisture collects on the evaporator and drains through the bottom of the case (through a drain pan for example). Once the vehicle is turned off, the evaporator warms up, and this warm, wet environment is highly conducive to fungal, mold, spore, bacterial, and protozoal growth. This problem of microorganism growth on the evaporator is especially common in areas of high humidity, where it is less likely that fluid on the evaporator will evaporate because of the high water content in the ambient air. And as evaporators incorporate more fins into their design and the distance between fins continues to decrease, the likelihood of trapping more moisture on the outside of the evaporator increases, as does the likelihood of increased microorganism growth.

The presence of microorganisms (most commonly bacteria, fungi, and protozoa) on the evaporator and other parts of the HVAC system is problematic for several reasons. First, these organisms produce foul odors that are transmitted into the passenger compartment. Second, the organisms themselves may be blown into the passenger compartment, leading to passenger infection (pneumonia, bronchitis) or other ailments. Allergic rhinitis and bronchial asthma in particular have both been shown to result from contamination of automobile air conditioners with microorganisms (“Respiratory allergies related to automobile air conditioners”, New England Journal of Medicine, 1984, Vol. 311, No. 25, pp. 1619-1621). Legionella bacteria and various molds are particularly worrisome. Third, the growth of microorganisms as well as the organic matter they produce (biofilm) may coat the heat exchange surfaces, reducing heat transfer while simultaneously increasing the pressure drop across the system. Hence, the growth of microorganisms in HVAC systems remains a considerable problem in motor vehicles.

Several detergents and fungicides are available to kill organisms that accumulate and grow on the evaporator. However, these chemicals are noxious and difficult to administer. A technician may need to wear a respirator while working with these chemicals. Moreover, gaining access to the evaporator can be time consuming and difficult, sometimes requiring extensive disassembly. In some cases, a hole must be drilled into the HVAC system, a hole that later has to be patched. Finally, since the chemicals eventually wear off, this procedure may have to be repeated at regular intervals, which is both inconvenient and expensive. Another approach is to install an after blow module (also known as an electronic evaporator dryer module), the purpose of which is to periodically operate the blower to dry off the evaporator once the vehicle is turned off. This is suboptimal since it requires energy use while the vehicle is turned off and does not kill the microorganisms. Moreover, both the blow module approach and application of chemicals merely prevent the growth of organisms within the HVAC system; they do not prevent transmission of infectious agents from entering from outside the car, nor do they cleanse the air from the passenger cabin of any potentially infectious agents.

Another problem with conventional motor vehicle HVAC systems is their inability to cleanse air from the passenger compartment of infectious pathogens. One might imagine several circumstances in which such a deficiency might lead passengers to become infected with an airborne pathogen, be it bacterial, viral, fungal (mold), spore, or other. One particularly worrisome organism is mycobacterium tuberculosis. Other organisms include various other bacteria and viruses that can lead to aerosolized transmission of the flu, colds, or other ailments. In the event that one person in a passenger cabin is infected (and perhaps is coughing or sneezing), the aerosolized infectious agent can be transmitted to other people in the closed cabin, leading to their infection. This is especially likely given the small space of the cabin. This becomes even more likely if the air is recirculated.

Finally, there is the potential problem of infectious agents from the outside environment entering the passenger cabin. These infectious agents include bacterial, viral, spore, fungal or other agents that may or may not be naturally present in the environment. With the increasing likelihood of the use of biological weapons, it would be of considerable benefit if automobiles and other motor vehicles were equipped with means of killing any infectious organisms that come in from the outside air. Such organisms might include but are not be limited to viruses such as small pox, bacteria such as anthrax, and various fungi/molds. Moreover, a system that inactivates airborne chemical compounds would also be of considerable interest. For example, a system that was able to inactivate a bioterrorism agent (including but not limited to nerve gas), allergen, or other noxious compound would be useful.

Current air filters in motor vehicle ventilation systems are ill suited to capture organisms below a certain size (<3 microns for example). As such, many infectious pathogens (including many bacteria and viruses) pass freely through the filters. Filters also have a tendency to become clogged, and need to be replaced periodically. Moreover, many motor vehicle owners tend to neglect recommended service actions; these filters may not always be replaced as required. Until they are replaced, clogged filters can lead to reduction in airflow.

Clearly there is a need for a system that solves at least one if not all of these three needs. The system should ideally prevent growth of microorganisms within the HVAC system (including on the evaporator), should eliminate infectious organisms that enter the HVAC system from the outside air, and should eliminate infectious organisms that enter the HVAC system from the passenger compartment (patient sneezing, coughing, breathing). Such a system should ideally be inexpensive, long-lasting, and not require an extensive redesign of current HVAC systems. Furthermore, the system should ideally be compact, and not add to the size of the current HVAC systems. Such a system would be an important contribution to the art.

### SUMMARY OF THE INVENTION

Systems involving the use of ultraviolet lighting and related elements to reduce the number of microorganisms within heating, ventilation, and air conditioning systems within motor vehicles are provided.

One non-limiting advantage of the present invention is that it provides a system and method that use ultraviolet light to reduce growth of microorganisms such as bacteria, fungi, spores, protozoa, and viruses within heating, ventilation, and air conditioning units within motor vehicles.

Another non-limiting advantage of the present invention is that it provides a system and method that use ultraviolet light to kill microorganisms such as bacteria, fungi, spores, protozoa, and viruses as they pass through heating, ventilation, and air conditioning units within motor vehicles.

According to one aspect of the present invention, an HVAC system is provided for a motor vehicle and includes at least one ultraviolet light source for treating microorganisms within the HVAC system.

According to another aspect of the present invention, a method for treating microorganisms within an HVAC system of a motor vehicle is provided and includes generating ultraviolet light within said HVAC system, effective to kill and prevent growth of microorganisms.

Additional advantages and features of the invention will appear from the following drawings and detailed description.

### BRIEF DESCRIPTION OF THE FIGURES

FIG. 1 provides a schematic diagram of a first embodiment of the invention in which ultraviolet light sources are located within outside and in-car air inlets, or within the drain pan of the HVAC system.

FIG. 2 provides a schematic diagram of a second embodiment of the invention in which an ultraviolet light source is located in the space between the air inlets and blower of the HVAC system.

FIG. 3 provides a schematic diagram of a third embodiment of the invention in which an ultraviolet light source is located between the blower and evaporator of the HVAC system.

FIG. 4 provides a schematic diagram of a fourth embodiment of the invention in which an ultraviolet light source is located near the evaporator of the HVAC system to provide light to the heat exchange surfaces of the evaporator.

FIG. 5 provides a schematic diagram of a fifth embodiment of the invention in which an ultraviolet light source is located between the evaporator and heater core of the HVAC system.

FIG. 6 provides a schematic diagram of a sixth embodiment of the invention in which an ultraviolet light source is located near the heater core of the HVAC system to provide light to the heat exchange surfaces of the heater core.

FIG. 7 provides a schematic diagram of a seventh embodiment of the invention in which an ultraviolet light source is located between the heater core and the outlets of the HVAC system.

FIG. 8 provides a schematic diagram of an eighth embodiment of the invention in which ultraviolet light sources are located within the outlets of the HVAC system.

### DETAILED DESCRIPTION OF THE EMBODIMENTS

Systems involving the use of ultraviolet lighting and related elements to reduce the number of microorganisms within heating, ventilation, and air conditioning systems within motor vehicles are provided. The term microorganism includes any organism (animal, plant, or other) of microscopic size. The term microorganism includes but is not limited to bacteria, fungi, molds, spores, protists, protozoa, algae, parasites, plants, viruses and prisons. The term motor vehicle includes any self-propelled wheeled conveyance that does not run on rails. The term motor vehicle includes but is not limited to automobiles, minivans, trucks, buses, vans, sport utility vehicles, rickshaws, recreational vehicles (RV), motor homes, or any vehicle with a passenger compartment set on wheels that are powered by a motor. Moreover, vehicles that are capable of being pulled by another vehicle with a motor, such as trailers and other transport vehicles are also included. Furthermore, while the preferred embodiments of the invention are described in connection with motor vehicles, it should be appreciated that the invention may be implemented in any vehicle employing an HVAC system.

Before the subject invention is further described, it is to be understood that the invention is not limited to the particular embodiments described below, as variations of the particular embodiments may be made and still fall within the scope of the invention. It is also to be understood that the terminology employed is for the purpose of describing particular embodiments, and is not intended to be limiting.

It must also be noted that as used in this specification, the singular forms “a”, “an”, and “the” include plural reference unless the context clearly indicates otherwise. Unless defined otherwise, all technical and scientific terms used herein have the same meaning as commonly understood to one of ordinary skill in the art to which this invention belongs.

As summarized above, the subject inventions are for use in reducing the number of and/or killing microorganisms such as bacteria, viruses, and fungi, spores, protozoa, and molds within HVAC systems within motor vehicles. An important advantage of implementing such a system is that it reduces passenger exposure to microorganisms and their byproducts as well as improves heat exchange efficiency and/or minimizes pressure drop within the system. A common feature of all the subject inventions is the use of ultraviolet light as a convenient means of preventing growth and/or transmission of microorganisms within the HVAC system.

Ultraviolet light may be used to kill microorganisms including bacteria, fungus/molds, spores, and viruses. For example, ultraviolet light has been used to purify air in hospitals, clean rooms, food processing plants, and office buildings. Ultraviolet light with wavelengths 240-280 nm has traditionally been used for this purpose. In particular, ultraviolet light at or near 254 nm has been shown to be particularly effective in killing microorganisms due to the resonance of this wavelength within molecular structures within cells. Exposure to ultraviolet light leads to cellular and/or genetic (DNA) damage to the cells and subsequent death. Even a short term exposure (seconds) to ultraviolet light can lead to effective killing of microorganisms.

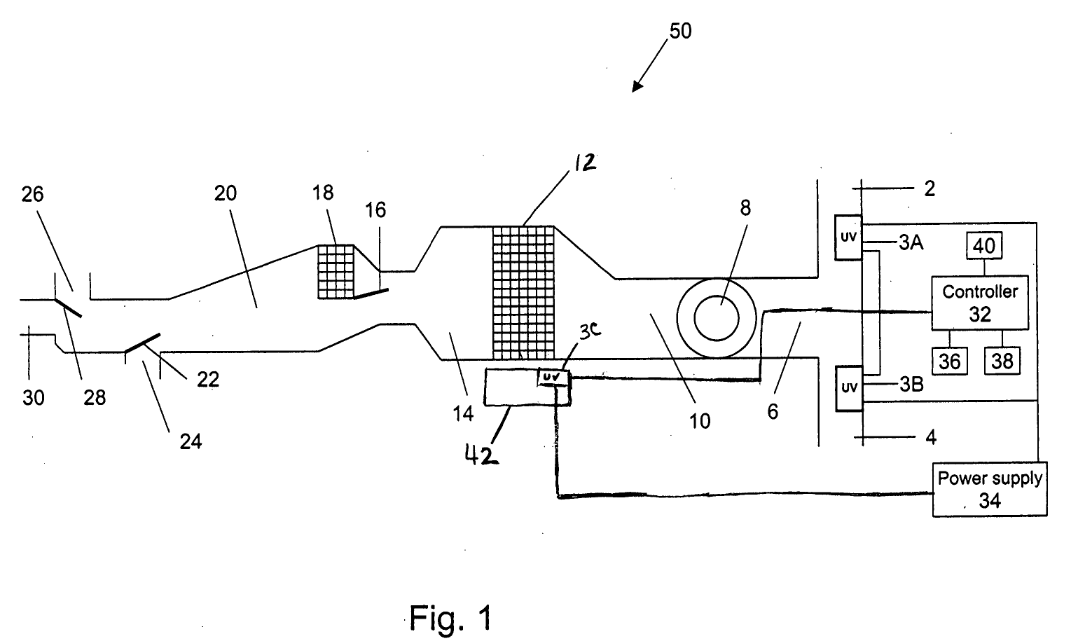

The present invention utilizes ultraviolet light within a motor vehicle HVAC system to substantially reduce or eliminate microorganisms within the system. Referring now to the figures, FIG. 1 illustrates a schematic diagram of a first embodiment of an HVAC system 50, including a plurality of ultraviolet light sources, which are located within the outside and in-car air inlets and/or within the drain pan of the HVAC system. HVAC system 50 includes an outside air inlet 2 and an in-car air inlet 4, which provide air from outside the car and air from inside the car (recirculated air), respectively, and ultraviolet light sources 3A and 3B, which are respectively disposed within inlets 2 and 4 and which provide ultraviolet light to the air inlets and surrounding conduits of the HVAC system. Positioning ultraviolet light sources 3A, 3B in these locations ensures that all air entering the system is subjected to ultraviolet light treatment. Additionally, HVAC components including parts of the air ducts/conduits are also exposed to ultraviolet light to kill microorganisms that might accumulate or grow in these areas.

HVAC system 50 further includes a blower 8 for circulating and/or transferring air within the system, pre-blower portion or conduit 6, post-blower portion or conduit 10, an evaporator 12, a drain pan 42, which may be located below the evaporator 12, post-evaporator portion or conduit 14, a temperature door 16, a heater core 18, post-heater core portion or conduit 20, air conditioning heater door 22, floor air outlet 24, defrost air outlet 26, air conditioning defrost door 28 and panel air outlet 30.

In the preferred embodiment, blower 8, evaporator 12, drain pan 42, heater core 18, and doors 16, 22 and 28 may comprise conventional and commercially available HVAC components. Furthermore, portion or conduits 6, 10, 14, and 20, inlets 2 and 4, and outlets 24, 26 and 30 may be formed from conventional materials (e.g., plastic, metal and the like) and may be shaped in a known manner to conform to the contained components and structure of a motor vehicle in which the HVAC system 50 is disposed.

In the preferred embodiment, each ultraviolet light source 3A, 3B may comprise a single ultraviolet light bulb, or a plurality of light bulbs placed next to one another in a linear, circumferential or other pattern. The ultraviolet light bulb itself may be of any convenient shape, including but not limited to spherical, linear, circumferential, or spiral. Commercially available bulbs from a variety of suppliers may be used, or the bulbs may be custom-shaped to conform to the internal portions of the HVAC conduits. The bulbs used may emit any wavelength or wavelengths of ultraviolet light, or some combination of ultraviolet light and non-ultraviolet light. Non-ultraviolet light includes but is not limited to visible and infrared light. Preferably, the ultraviolet light will be in the UV-C spectrum, and most preferably there will be ultraviolet light with a wavelength at or near 254 nm, which has been shown to be particularly effective in killing microorganisms. In certain embodiments, bulb(s) which almost exclusively emit ultraviolet light above a certain wavelength (>200 nm or >220 nm for example) may be used, since they offer the additional benefit of producing less ozone.

The ultraviolet light sources 3A, 3B may be communicatively coupled to a conventional controller 32, which is effective to selectively activate and deactivate the light sources 3A, 3B according to a predetermined control strategy. Particularly, the controller 32 may cause the light sources 3A, 3B to be selectively connected to and disconnected from a power supply 34, which may comprise the motor vehicle's battery, the vehicle's alternator, an external generator, a solar generator, a fuel cell, or other suitable power supply, or any combination of the foregoing power sources.

The controller 32 may be a conventional microprocessor-based controller operating under stored program control. The controller 32 may be communicatively coupled to conventional vehicle components and accessories 36 (e.g., the ignition switch, air conditioning unit, fan, HVAC system and the like), switches 38 and/or sensors 40, which may be monitored by controller 32 in order to control the operation of the light sources. For example, in certain embodiments, the ultraviolet light sources 3A, 3B may be activated or turned on whenever the engine is running. In other embodiments, the ultraviolet light sources 3A, 3B may be activated when the engine is turned off, and may remain on for a predetermined period of time (for example 1-100 minutes) or until the engine is turned on again. In still other embodiments, the ultraviolet light sources 3A, 3B may be turned on when the air conditioning unit is started, when the fan is started, or when the HVAC system is turned on. In other embodiments, the ultraviolet light sources 3A, 3B may be turned on when the air conditioning unit is turned off, when the fan is turned off, or when the HVAC system is turned off. In still other embodiments, the ultraviolet light sources 3A, 3B are always activated. In other embodiments, the ultraviolet light sources 3A, 3B are turned on and off periodically using a timing mechanism or strategy. Furthermore, in other embodiments, the unit can be turned on or off manually by the user through a switch 38 or other mechanism that can be controlled from the passenger compartment. This mechanism may or may not include an automatic timer for activating or deactivating the ultraviolet light sources 3A, 3B after a predetermined period of time. In other embodiments, the ultraviolet light sources 3A, 3B are selectively activated and deactivated in an intermittent manner by a timer. Finally, there may be a switch or mechanism (e.g., switch 38) that turns the light source off indefinitely.

The system may further comprise sensors 40 for sensing certain conditions and communicating signals to controller 32 for either turning the ultraviolet light sources 3A, 3B on or off, based on those conditions. Sensors 40 may be disposed inside or outside the HVAC system. These certain conditions that may be detected by the sensor include but are not limited to humidity, barometric pressure, and temperature. Other types of sensors include biologic sensors that are capable of detecting various organisms or their byproducts inside the passenger compartment, outside the vehicle, inside the HVAC system, etc. These organisms may include but are not limited to bacteria, molds, fungi, spores, protozoa, and viruses. Other types of sensors include chemical sensors that can detect various chemical compounds or materials, noxious or otherwise. In certain embodiments, the sensors 40 will feed information to the control system 32 which will selectively activate and deactivate ultraviolet light sources. Alternatively, the sensors 40 may be used to alert the user/passenger of a potential need to turn on or turn off the ultraviolet light source, and she may then do so manually by use of a switch 38.

In some embodiments, there is an ultraviolet light source 3C, which may be substantially identical in structure and operation to light sources 3A and 3B, placed within, above or in relative close proximity to the drain pan 42 of the HVAC system to limit the growth of organisms in the drain pan and nearby areas.

In certain embodiments, there is a sensor in the passenger compartment or in a portion of the HVAC system distant from the light source and/or near the passenger compartment (in the outlets for example) that detects ultraviolet light, ozone, or other product or byproduct of this invention. If the sensor detects a particular compound or byproduct, it triggers an alarm (visual, audio or other). In some embodiments this alarm will cause the ultraviolet light system to turn off. Thus, the sensor helps to prevent transmission of ultraviolet light, ozone, or other compound into the passenger compartment.

In some embodiments, an ultraviolet light filtering mechanism may be placed between the ultraviolet light source and the passenger or other inner compartment that filters out any ultraviolet light that might otherwise reach the passenger compartment. In other embodiments, the presence of surfaces or materials that absorb or do not reflect ultraviolet light may be present in the ducts, portions or conduits between the ultraviolet light source and the passenger compartment. This serves to prevent transmission of ultraviolet light into the passenger or other inner compartment of the vehicle.

In certain embodiments, the inner surfaces of some if not all components of the HVAC system are coated with or are made of substances that reflect ultraviolet light. The use of such reflective materials/coating is aimed at providing continued exposure to reflected ultraviolet light at locations distant from the light source. This continued exposure to ultraviolet light makes it more likely that organisms in the air will be killed during their passage time in the HVAC system. The materials may include any conventional and commercially available materials that reflect ultraviolet light. These include but are not limited to magnesium oxide, polished aluminum sheet, white plaster, aluminum paint, chromium, nickel and stainless steel. By using such reflective surfaces, it is possible to prevent organism growth everywhere in the HVAC system. Moreover, because reflected ultraviolet light increases killing efficiency, fewer light sources may be required to achieve a desired kill rate.

In some embodiments, there may be a device located within the HVAC system that reduces the amount of ozone, if any, which may be produced during the ultraviolet light generation process.

In some embodiments, there may be one or more conventional air filtering devices present within the HVAC system. The one or more filtering devices may be disposed and/or incorporated within any of the conduits or components of the HVAC system. Such air filtering devices would be useful in reducing the amount of microorganisms or debris in the air within the HVAC system. Alternatively, the filter(s) (e.g., containing activated carbon or other compounds) could serve to remove foul odors in the air. The combination of the filter(s) and the ultraviolet light exposure would provide a complementary solution to both remove/kill microorganisms as well as their odors.

In some embodiments, when the ultraviolet light source is turned on, the HVAC unit reverts to “recirculation mode” in which the outside air inlet 2 is closed and the recirculated air inlet 4 is open. As the air is recirculating, microorganisms are exposed to ultraviolet light multiple times as they pass through the system, increasing the likelihood that they will be killed.

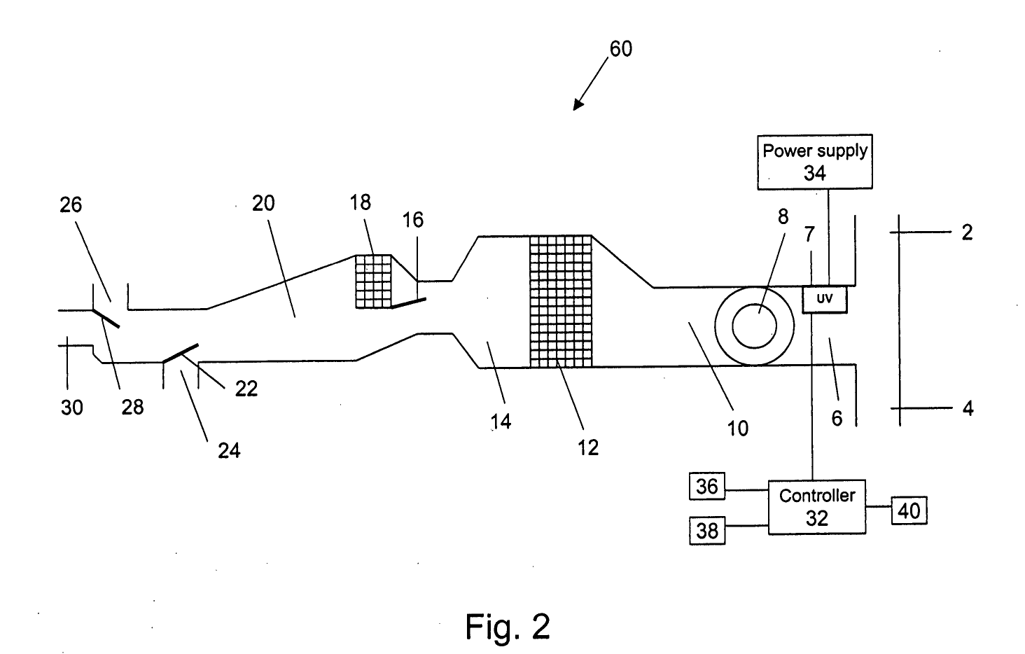

FIG. 2 provides a schematic diagram of a second embodiment of an HVAC system 60 in which an ultraviolet light source 7 is located in pre-blower portion or conduit 6, which is defined as the space between air inlets 2 and 4 and blower 8. Ultraviolet light source 7 may be substantially identical in structure and operation to light sources 3A and 3B. Positioning ultraviolet light in this location ensures that all air entering the system is subjected to ultraviolet light prior to reaching blower 8. Additionally, HVAC components including parts of the blower and the air ducts/conduits are also exposed to ultraviolet light. This second embodiment is substantially identical in structure and function as HVAC system 50 shown in FIG. 1, with like elements bearing identical reference numerals.

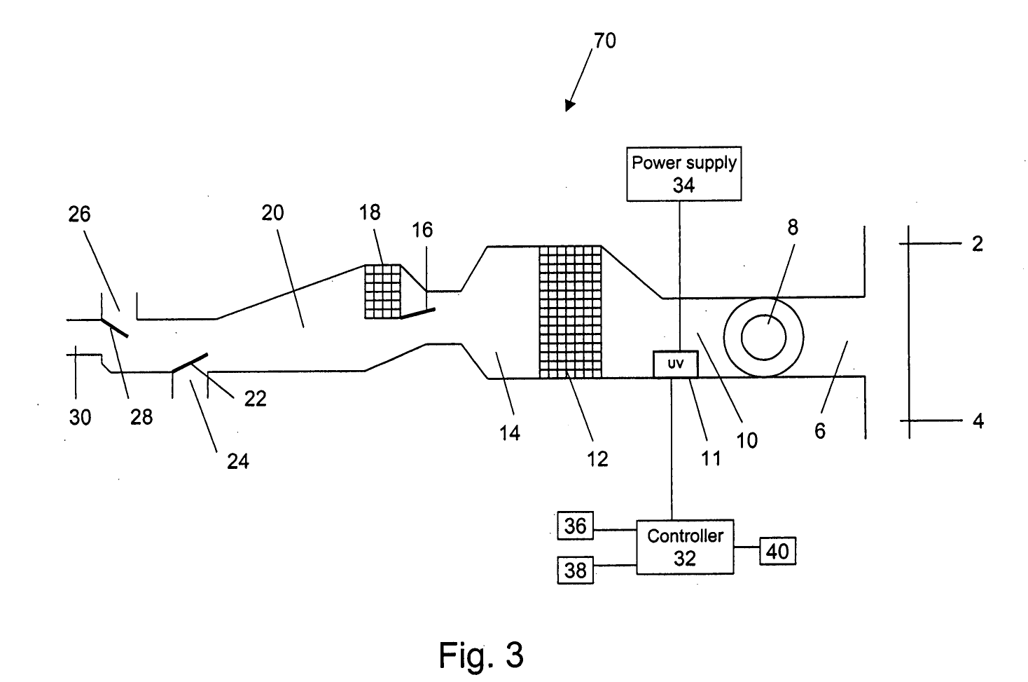

FIG. 3 provides a schematic diagram of a third embodiment of an HVAC system 70 in which an ultraviolet light source 11 is located in post-blower portion or conduit 10, which is defined as the space between blower 8 and evaporator 12. Ultraviolet light source 11 may be substantially identical in structure and operation to light sources 3A and 3B. Positioning an ultraviolet light source in this location ensures that all air propelled into post-blower portion or conduit 10 is subjected to ultraviolet light. Additionally, HVAC components including parts of the blower, evaporator, and air ducts/conduits are also exposed to ultraviolet light. This third embodiment is substantially identical in structure and function as HVAC system 50 shown in FIG. 1, with like elements bearing identical reference numerals.

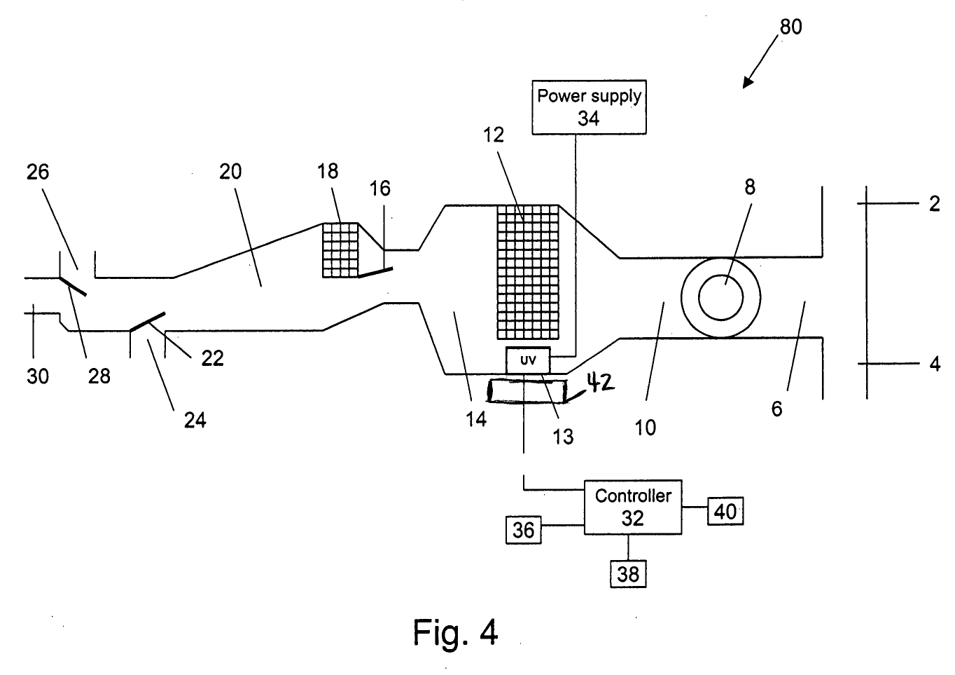

FIG. 4 provides a schematic diagram of a fourth embodiment of an HVAC system 80 in which an ultraviolet light source. 13 is located in close proximity to evaporator 12, allowing ultraviolet light exposure to parts of the evaporator (including heat exchange surfaces) and surrounding components such as the drain pan 42, etc. Ultraviolet light source 13 may be substantially identical in structure-and operation to light sources 3A and 3B. Positioning an ultraviolet light source in this location ensures that air propelled through evaporator 12 is subjected to ultraviolet light treatment. Additionally, HVAC components including parts of evaporator 12 and air ducts/conduits are also exposed to ultraviolet light. By exposing the heat exchange surfaces to ultraviolet light, the surfaces will be less prone to microorganism/biofilm buildup with a corresponding increase in heat exchange efficiency. This fourth embodiment is substantially identical in structure and function as HVAC system 50 shown in FIG. 1, with like elements bearing identical reference numerals.

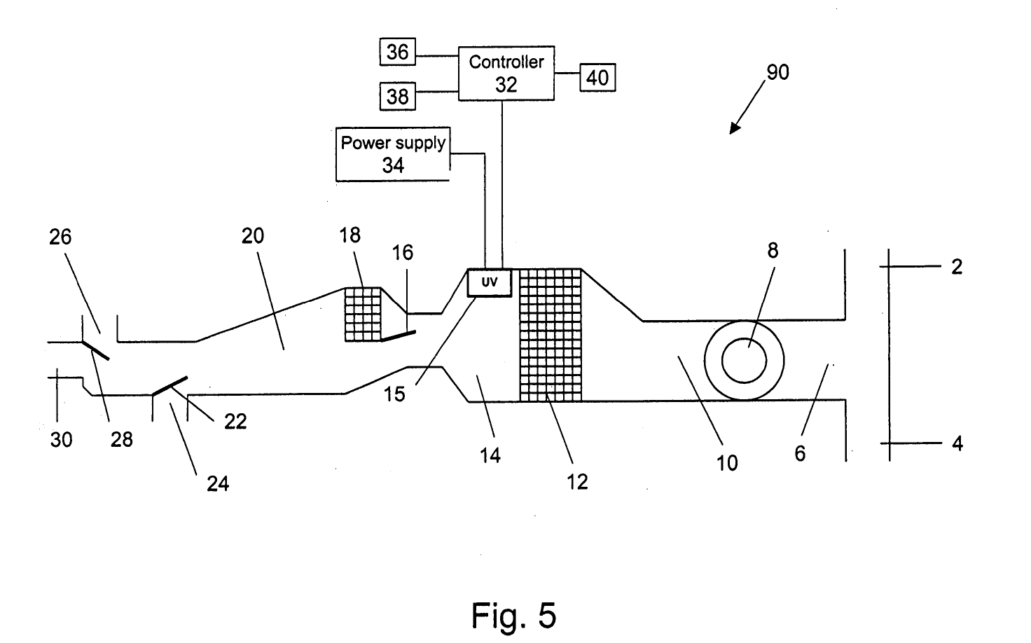

FIG. 5 provides a schematic diagram of a fifth embodiment of an HVAC system 90 in which an ultraviolet light source 15 is located in post-evaporator portion or conduit 14, which is defined as the space between evaporator 12 and heater core 18. Ultraviolet light source 15 may be substantially identical in structure and operation to light sources 3A and 3B. Positioning an ultraviolet light source in this location ensures that all air propelled into the post-evaporator portion or conduit 14 is exposed to ultraviolet light. Additionally, HVAC components including parts of evaporator 12 and air ducts/conduits are also exposed to ultraviolet light. This fifth embodiment is substantially identical in structure and function as HVAC system 50 shown in FIG. 1, with like elements bearing identical reference numerals.

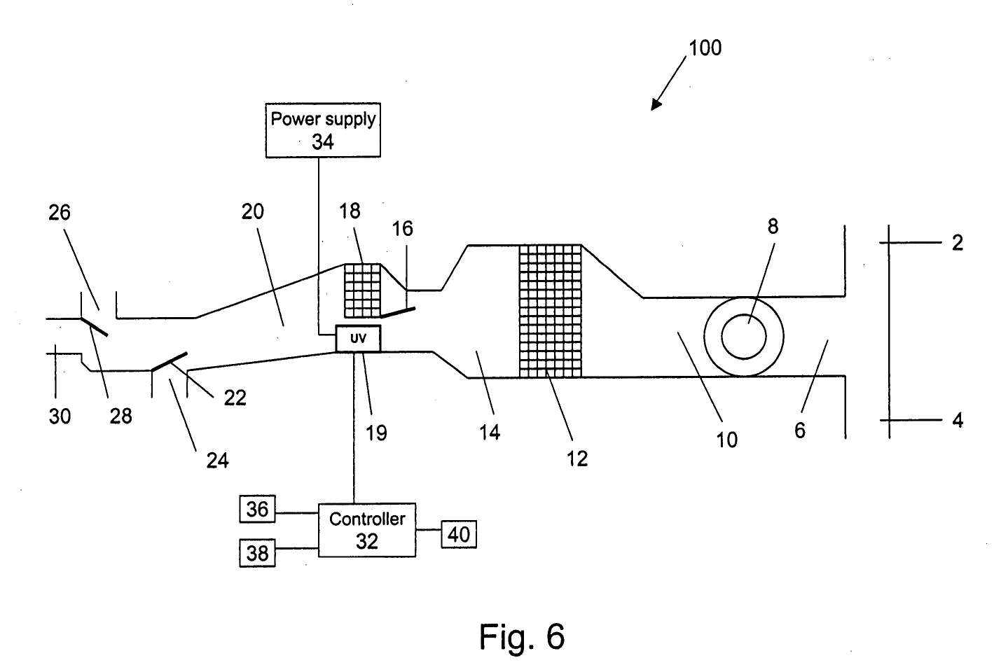

FIG. 6 provides a schematic diagram of a sixth embodiment of an HVAC system 100 in which ultraviolet light source 19 is located in close proximity to heater core 18, allowing ultraviolet light exposure to parts of the heater core (including heat exchange surfaces) and surrounding components. Ultraviolet light source 19 may be substantially identical in structure and operation to light sources 3A and 3B. Positioning an ultraviolet light source in this location ensures that air propelled through heater core 18 is subjected to ultraviolet light treatment. Additionally, HVAC components including parts of heater core 18, air ducts/conduits, and temperature door 16 are also exposed to ultraviolet light. By exposing the heat exchange surfaces to ultraviolet light, it is expected that the surfaces will be less prone to microorganism/biofilm buildup with a corresponding increase in heat exchange efficiency. This sixth embodiment is substantially identical in structure and function as HVAC system 50 shown in FIG. 1, with like elements bearing identical reference numerals.

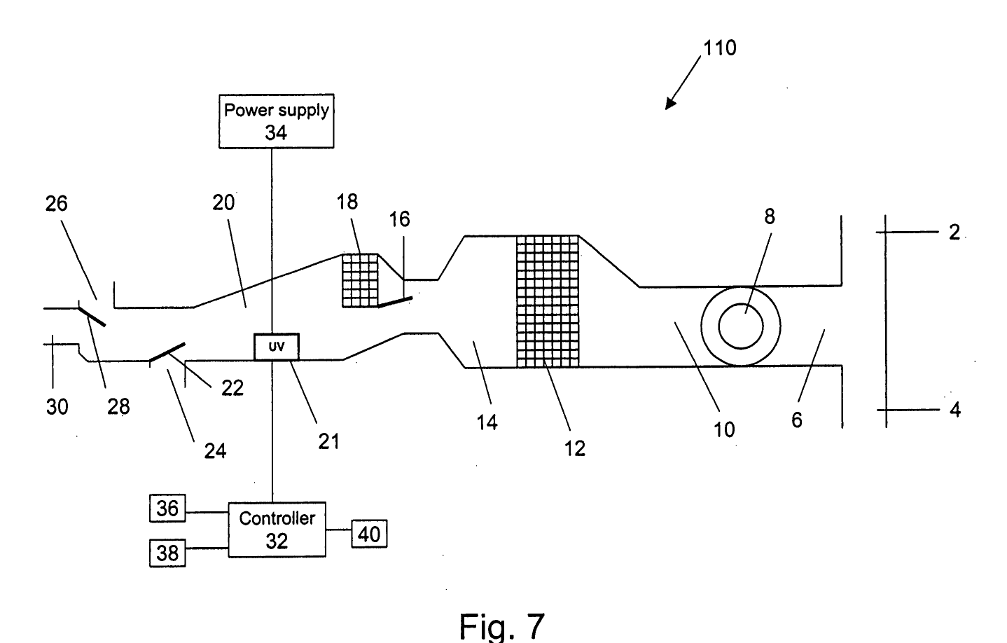

FIG. 7 provides a schematic diagram of a seventh embodiment of an HVAC system 110 in which ultraviolet light source 21 is operatively disposed in the post-heater core portion or conduit 20, which is defined as the space between heater core 18 and the outlets downstream. Ultraviolet light source 21 may be substantially identical in structure and operation to light sources 3A and 3B. Positioning an ultraviolet light source in this location ensures that all air propelled into post-heater core portion or conduit 20 is exposed to ultraviolet light. Additionally, HVAC components including parts of heater core 18 and the air ducts/conduits are also exposed to ultraviolet light. This seventh embodiment is substantially identical in structure and function as HVAC system 50 shown in FIG. 1, with like elements bearing identical reference numerals.

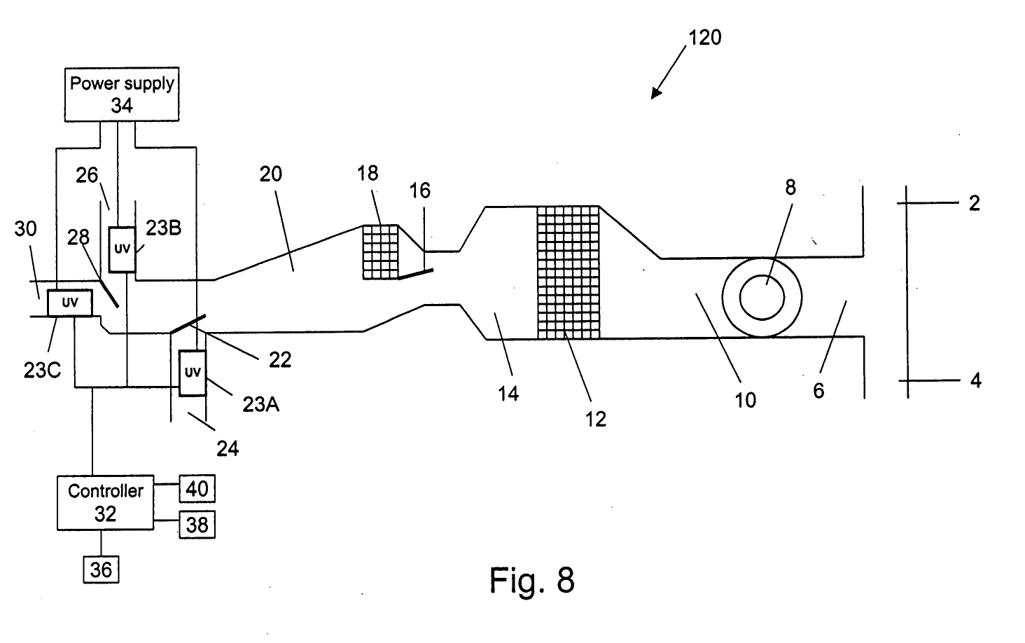

FIG. 8 provides a schematic diagram of an eighth embodiment of an HVAC system 120 in which ultraviolet light sources are located in one or more air outlets. The diagram in FIG. 8 illustrates three ultraviolet light sources 23A, 23B, and 23C, which are respectively and operatively disposed in floor air outlet 24, defrost air outlet 26, and panel air outlet 30. All three of these outlets provide air into the passenger compartment albeit in different locations. Ultraviolet light sources 23A, 23B, and 23C provide ultraviolet light into their respective air outlets and onto surrounding portions of the HVAC system including A/C heater door 22 and A/C defrost door 28. Ultraviolet light sources 23A, 23B and 23C may be substantially identical in structure and operation to light sources 3A and 3B. Positioning ultraviolet light sources in these locations ensures that all air exiting the system is subjected to ultraviolet light treatment. Additionally, HVAC components including parts of the air ducts/conduits are also exposed to ultraviolet light. This eighth embodiment is substantially identical in structure and function as HVAC system 50 shown in FIG. 1, with like elements bearing identical reference numerals.

Finally, in other embodiments, ultraviolet light sources may be placed in multiple locations, combining elements from two or more of the previously detailed embodiments. For example, ultraviolet light sources may be placed in both pre-blower portion or conduit 6 and post-blower portion or conduit 10. Any combination of locations that have been described previously fall within the scope and spirit of this application.

Hence, it is evident that the subject devices provide a number of important advantages over prior art devices. The use of ultraviolet light in HVAC systems in motor vehicles offers the benefit of killing microorganisms (bacteria, viruses, molds, spores, protozoa, and fungi) that enter from the outside air, preventing passengers within the passenger compartment from being exposed to these potentially harmful pathogens. Moreover, ultraviolet light exposure serves to kill microorganisms that are already present in the passenger compartment (for example, by an already infected passenger who might be coughing or sneezing). Finally, ultraviolet light exposure to components within the HVAC system (including the evaporator) offers the benefit of reducing microorganism growth on the heat exchange surfaces, therefore improving the efficiency of the system. Simultaneously, such a reduction in microorganism growth is expected to reduce the incidence of respiratory ailments such as allergic rhinitis and bronchial asthma since these microorganisms are not blown into the passenger compartment. Moreover, killing microorganisms also prevents transmission of foul odors. As such, the subject invention represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of the invention.

What is claimed is:

1. An HVAC system for a motor vehicle comprising at least one ultraviolet light source for treating microorganisms within the HVAC system.

2. The HVAC system of claim 1 wherein the at least one ultraviolet light source comprises a bulb for emitting ultraviolet light in the UV-C spectrum, effective to kill microorganisms.

3. The HVAC system of claim 2 wherein the ultraviolet light emitted by the at least one ultraviolet light source is approximately 254 nm in wavelength.

4. The HVAC system of claim 1 further comprising a controller which is communicatively coupled to the at least one ultraviolet light source for selectively activating and deactivating the at least one ultraviolet light source.

5. The HVAC system of claim 4 further comprising at least one sensor for monitoring a parameter, the sensor being communicatively coupled to the controller and adapted to provide signals to the controller for selectively activating and deactivating the at least one ultraviolet light source.

6. The HVAC system of claim 1 wherein the at least one ultraviolet light source is operatively disposed within at least one air inlet of the HVAC system.

7. The HVAC system of claim 1 wherein the at least one ultraviolet light source is operatively disposed within a pre-blower portion of the HVAC system.

8. The HVAC system of claim 1 wherein the at least one ultraviolet light source is operatively disposed within a post-blower portion of the HVAC system.

9. The HVAC system of claim 1 wherein the at least one ultraviolet light source is operatively disposed in relative close proximity to an evaporator of the HVAC system.

10. The HVAC system of claim 1 wherein the at least one ultraviolet light source is operatively disposed within a post-evaporator portion of the HVAC system.

11. The HVAC system of claim 1 wherein the at least one ultraviolet light source is operatively disposed in relative close proximity to a heater core of the HVAC system.

12. The HVAC system of claim 1 wherein the at least one ultraviolet light source is operatively disposed within a post-heater core portion of the HVAC system.

13. The HVAC system of claim 1 wherein the at least one ultraviolet light source is operatively disposed within at least one air outlet of the HVAC system.

14. The HVAC system of claim 1 wherein the at least one ultraviolet light source is operatively disposed in relative close proximity to a drain pan of the HVAC system.

15. The HVAC system of claim 1 further comprising a plurality of conduits for communicating air to an inner compartment of the vehicle, the conduits having inner surfaces that absorb ultraviolet light, thereby preventing the transmission of ultraviolet light into the inner compartment.

16. The HVAC system of claim 1 further comprising a plurality of conduits for communicating air within the vehicle, the conduits having inner surfaces that reflect ultraviolet light, thereby increasing dispersion of ultraviolet light within the HVAC system.

17. A method for treating microorganisms within an HVAC system of a motor vehicle comprising:
generating ultraviolet light within said HVAC system, effective to kill and prevent growth of microorganisms.

18. The method of claim 17 wherein the ultraviolet light is in the UV-C spectrum.

19. The method of claim 18 wherein the ultraviolet light is approximately 254 nm in wavelength.

20. The method of claim 17 wherein the ultraviolet light is generated by at least one ultraviolet light source which is selectively activated and deactivated according to a control strategy.

21. The method of claim 20 further comprising:
monitoring a parameter; and
selectively activating and deactivating the at least one ultraviolet light source based upon the parameter.

22. The method of claim 17 wherein the ultraviolet light is generated in at least one air inlet of the HVAC system.

23. The method of claim 17 wherein the ultraviolet light is generated within a pre-blower portion of the HVAC system.

24. The method of claim 17 wherein the ultraviolet light is generated within a post-blower portion of the HVAC system.

25. The method of claim 17 wherein the ultraviolet light is generated in relative close proximity to an evaporator of the HVAC system.

26. The method of claim 17 wherein the ultraviolet light is generated within a post-evaporator portion of the HVAC system.

27. The method of claim 17 wherein the ultraviolet light is generated in relative close proximity to a heater core of the HVAC system.

28. The method of claim 17 wherein the ultraviolet light is generated within a post-heater core portion of the HVAC system.

29. The method of claim 17 wherein the ultraviolet light is generated within at least one air outlet of the HVAC system.

30. The method of claim 17 wherein the at least one ultraviolet light is generated in relative close proximity to a drain pan of the HVAC system.
